(19) United States
(12) Patent Application Publication
(10) Pub. No.: US 2021/0161813 A1
(43) Pub. Date: Jun. 3, 2021

(54) COMESTIBLE COMPOSITIONS FOR PROMOTING ATP SYNTHESIS

(71) Applicant: Maine Natural Health Company, Inc., Warren, ME (US)

(72) Inventors: Harry J. LEIGHTON, Warren, ME (US); Kenneth CARTER, Marina Del Rey, CA (US)

(21) Appl. No.: 17/171,811

(22) Filed: Feb. 9, 2021

Related U.S. Application Data
(63) Continuation of application No. PCT/US2019/045973, filed on Aug. 9, 2019.
(60) Provisional application No. 62/717,243, filed on Aug. 10, 2018.

(51) Int. Cl. A61K 9/00; A61K 31/221; A61K 31/4745; A61K 31/375; A61K 31/355; A61K 31/015; A61K 33/04; A61K 31/455; A61K 31/4415; A61K 31/714; A61K 31/706; A61K 47/12; A61K 47/02; A61K 31/122; A61K 31/09; A61K 31/7004; A61P 43/00

(52) U.S. Cl. CPC A61K 9/0095; A61P 43/00; A61K 31/4745; A61K 31/375; A61K 31/355; A61K 31/015; A61K 33/04; A61K 31/455; A61K 31/4415; A61K 31/714; A61K 31/706; A61K 47/12; A61K 47/02; A61K 31/122; A61K 31/09; A61K 31/7004; A61K 31/221

(57) ABSTRACT

Comestible compositions including a mitochondrial anti-oxidant, a fatty acid transport facilitator, and a substrate for ATP synthesis are disclosed. For example, a comestible composition may include D-ribose, acetyl-L-carnitine, and coenzyme Q10. Also disclosed are kits including the comestible compositions and methods of use of the comestible compositions.

### FIELD OF THE INVENTION

The invention relates to the field of nutraceuticals and molecular medicine. More specifically, the invention provides comestible compositions, methods of their preparation, and methods of their use (e.g., to provide a health benefit, such as to treat a disease disorder or condition).

### BACKGROUND

Mitochondria are the key organelle responsible for cellular energy production in the form of adenosine triphosphate (ATP) through a process called oxidative phosphorylation. Aging and various chronic disease states result in a loss of mitochondrial number and impact the efficiency of the remaining mitochondria to synthesis ATP. Ultimately as energy declines, mitochondria undergo apoptosis, leading eventually to cell death. Cumulative cell death leads to organ system failure and ultimately the death of the individual. Mitochondrial loss and an overall decrease in efficiency of synthesizing ATP is potentially the root cause of aging.

Mitochondrial dysfunction is associated with virtually all chronic diseases and the aging process. Mitochondrial dysfunction is a loss in the efficiency of ATP synthesis. The rate of ATP synthesis, required for cells to operate optimally is compromised. In general mitochondrial dysfunction results from an inadequate number of mitochondria.

Mitochondrial dysfunction results in loss of energy and increasing fatigue and a loss of alertness that impacts cognition and learning. These symptoms are often the result of inadequate ATP to maintain healthy cell functions (maintaining ion gradients, enzyme synthesis and activity, muscle synthesis and contractility, heart function and neuronal function), leading to numerous chronic diseases, including: Alzheimer's Disease, Parkinson's Disease, Huntington's Disease, amyotrophic lateral sclerosis, cardiovascular disease including congestive heart failure and atherosclerosis, metabolic diseases such as diabetes, neuroendocrine disorders, multiple sclerosis, psychiatric diseases such as depression, autism, bipolar syndromes, schizophrenia, chronic fatigue syndromes, Gulf War illnesses, fibromyalgia, and chronic muscle atrophy. In fact it is generally believed that nearly all diseases are impacted by mitochondrial dysfunction.

To our knowledge, no one chemical substance has been shown to prevent mitochondrial dysfunction or to enhance mitochondrial activation and/or biogenesis. Exercise has been shown to be beneficial to mitochondrial biogenesis.

There is a need for comestible compositions capable of promoting ATP synthesis.

### SUMMARY OF THE INVENTION

In general, the invention provides comestible compositions (e.g., nutraceutical compositions and dietary supplements), methods of their preparation, and methods of their use (e.g., to provide a health benefit). Without wishing to be bound by theory, the compositions of the invention may promote adenosine triphosphate (ATP) synthesis, thereby providing a health benefit, as described herein. The combination of components (e.g., the combination of a substrate for ATP synthesis, a mitochondrial anti-oxidant, and a fatty acid transport facilitator) may be synergistic in its effect on ATP synthesis and the associated health benefits.

In one aspect, the invention provides a comestible composition including a substrate for ATP synthesis, a mitochondrial anti-oxidant, and a fatty acid transport facilitator.

In some embodiments, the weight ratio of the substrate for ATP synthesis to the mitochondrial anti-oxidant is 25:1 to 100:1. In certain embodiments, the weight ratio of the substrate for ATP synthesis to the mitochondrial anti-oxidant is 50:1. In particular embodiments, the weight ratio of the fatty acid transport facilitator to the mitochondrial anti-oxidant is 5:2 to 10:1. In further embodiments, the weight ratio of the fatty acid transport facilitator to the mitochondrial anti-oxidant is 5:1. In yet further embodiments, the weight ratio of the substrate for ATP synthesis to the fatty acid transport facilitator is 5:2 to 20:1. In still further embodiments, the weight ratio of the substrate for ATP synthesis to the fatty acid transport facilitator is 10:1. In other embodiments, the weight ratio of the substrate for ATP synthesis to the fatty acid transport facilitator to the mitochondrial anti-oxidant is 50:5:1.

In yet other embodiments, the composition is formulated for administration once daily, twice daily, or three times daily. In still other embodiments, the composition includes 5 g to 16.5 g of the substrate for

ATP synthesis per serving.

In particular embodiments, the composition is formulated for administration three times daily, and the composition includes, e.g., 5 g to 5.5 g of the substrate for ATP synthesis per serving. In certain embodiments, the composition is formulated for administration twice daily, and the composition includes, e.g., 7.5 g to 8.3 g of the substrate for ATP synthesis per serving. In some embodiments, the composition is formulated for administration once daily, and the composition includes, e.g., 15 g to 16.5 g of the substrate for ATP synthesis per serving. In further embodiments, the composition includes, e.g., 500 mg to 1.65 g of acetyl-L-carnitine per serving. In yet further embodiments, the composition is formulated for administration three times daily, and the composition includes, e.g., 500 mg to 550 mg of the fatty acid transport facilitator per serving. In still further embodiments, the composition is formulated for administration twice daily, and the composition includes, e.g., 750 mg to 825 mg of the fatty acid transport facilitator per serving. In other embodiments, the composition is formulated for administration once daily, and the composition includes, e.g., 1.5 g to 1.65 g of the fatty acid transport facilitator per serving. In yet other embodiments, the composition includes, e.g., 100 mg to 300 mg of the mitochondrial anti-oxidant per serving. In still other embodiments, the composition is formulated for administration three times daily, and the composition includes, e.g., 100 mg to 110 mg of the mitochondrial anti-oxidant per serving. In some embodiments, the composition is formulated for administration twice daily, and the composition includes, e.g., 200 mg to 220 mg of the mitochondrial anti-oxidant per serving. In certain embodiments, the composition is formulated for administration once daily, and the composition includes, e.g., 300 mg to 330 mg of the mitochondrial anti-oxidant per serving.

In certain embodiments, the composition further includes an antioxidant (e.g., pyrroloquinoline quinone (PQQ), vitamin C, vitamin E, β-carotene, a polyphenol, or an inorganic selenium). In some embodiments, the composition is formulated to provide the antioxidant at a dose of 1 to 100 mg/day.

In further embodiments, the composition further includes one or more Vitamins B (e.g., Vitamin B3, Vitamin B6, or Vitamin B12). In yet further embodiments, the one or more Vitamins B are present in an amount corresponding to 100% to 1000% of the recommended dietary allowance.

In still further embodiments, the composition further includes NAD or an NAD precursor (e.g., nicotinamide riboside).

In some embodiments, the composition further includes a flavoring. In particular embodiments, the composition includes 2 to 2.5 grams of flavoring per serving. In certain embodiments, the flavoring is a citrus punch flavoring or mojito flavoring. In further embodiments, the flavoring includes citric acid.

In particular embodiments, the composition further includes a sodium salt. In certain embodiments, the composition includes the sodium salt in an amount providing 200 mg to 800 mg of Na⁺ per serving. In some embodiments, the composition includes the sodium salt in an amount providing 200 mg to 240 mg of Na⁺ per serving, and the composition is formulated, e.g., for three times daily administration. In further embodiments, the composition includes the sodium salt in an amount providing 300 mg to 400 mg of Na⁺ per serving, and the composition is formulated for, e.g., twice daily administration. In yet further embodiments, the composition includes the sodium salt in an amount providing 600 mg to 800 mg of Na⁺ per serving, and the composition is formulated for, e.g., once daily administration.

In certain embodiments, the composition further includes a potassium salt. In some embodiments, the composition includes the potassium salt in an amount providing 150 mg to 500 mg of K⁺ per serving. In particular embodiments, the composition includes the potassium salt in an amount providing 150 mg to 165 mg of K⁺ per serving, and the composition is formulated for, e.g., three times daily administration. In further embodiments, the composition includes the potassium salt in an amount providing 225 mg to 250 mg of K⁺ per serving, and the composition is formulated for, e.g., twice daily administration. In yet further embodiments, the composition includes the potassium salt in an amount providing 450 mg to 500 mg of K⁺ per serving, and the composition is formulated for, e.g., once daily administration.

In some embodiments, the composition further includes silicon dioxide. In certain embodiments, the composition includes 1% to 2% (w/w) of silicon dioxide.

In particular embodiments, the mitochondrial anti-oxidant is coenzyme Q10. In further embodiments, the mitochondrial anti-oxidant is ubiquinol 10.

In certain embodiments, the fatty acid transport facilitator is acetyl-L-carnitine. In particular embodiments, the fatty acid transport facilitator is propionyl-L-carnitine. In further embodiments, the fatty acid transport facilitator is isovaleryl-L-carnitine.

In some embodiments, the substrate for ATP synthesis facilitator is D-ribose.

In further embodiments, the composition is a powder. In yet further embodiments, the composition is a beverage (e.g., a hydration beverage).

In another aspect, the invention provides a kit including the comestible composition of the invention and instructions to mix one serving of the composition with water to produce a beverage. In some embodiments, instructions include an instruction to mix one serving of the composition with 12 US fl oz of water. In particular embodiments, the instructions further include instructions to consume the beverage within 1 hour of the mixing step. In certain embodiments, the instructions further include instructions to consume the beverage once daily, twice daily, or three times daily.

In yet another aspect, the invention provides a method of preparing the beverage composition of the invention (e.g., a hydration beverage) including mixing the comestible composition of the invention in the powder form with water. In some embodiments, the comestible composition of the invention in the powder form is mixed with about 12 US fl oz of water.

In still another aspect, the invention provides a method of providing a health benefit to a subject, the method including administering the comestible composition of the invention to the subject. In some embodiments, the subject is in need of the health benefit.

In particular embodiments, the health benefit includes an athletic performance enhancement.

In certain embodiments, the health benefit includes reduction in the recovery times following an intense period of exercise. In further embodiments, the intense period of exercise is running a marathon, weight lifting, participation in a game of soccer, participation in a game of rugby, or participation in a game of football.

In some embodiments, the health benefit includes an energy state improvement or maintenance during a long period in the field. In particular embodiments, the long period in the field is a military operation or a long hike.

In further embodiments, the health benefit includes improvement of alertness in the subject.

In yet further embodiments, the health benefit includes enhancement of muscle protein synthesis in the subject during a period of immobility.

In a further aspect, the invention provides a method of treating a subject in need thereof, the method including administering the comestible composition of the invention to the subject.

In particular embodiments, the subject suffers from a disease, disorder, or condition associated with mitochondrial dysfunction, and the step of administering treats the disease, disorder, or condition. In certain embodiments, the subject suffers from a disease, disorder, or condition associated with an inadequate production of ATP, and the step of administering treats the disease, disorder, or condition.

In some embodiments, the subject suffers from a disease, disorder, or condition selected from the group consisting of ischemic heart disease, congestive heart failure, chronic fatigue syndrome, and peripheral arterial disease, and the step of administering treats the disease, disorder, or condition.

In certain embodiments, the subject suffers from a disease, disorder, or condition selected from the group consisting of amyotrophic lateral sclerosis, myasthenia, diabetes, Parkinson's disease, Huntington's disease, and Alzheimer's disease, and the step of administering treats the disease, disorder, or condition. In further embodiments, the disease, disorder, or condition is Parkinson's disease. In yet further embodiments, the step of administering slows the degeneration of dopaminergic neurons in the subject. In still further embodiments, the disease, disorder, or condition is Alzheimer's disease. In other embodiments, the step of administering slows the degeneration of cholinergic neurons in the subject.

In particular embodiments, the subject suffers from hypothyroidism associated with energy and metabolic deficiencies, and the step of administering treats hypothyroidism.

In some embodiments, the subject suffers from wasting disease secondary to cancer, and the step of administering treats wasting disease secondary to cancer. In particular embodiments, the wasting disease is secondary to pancreatic cancer. In certain embodiments, the subject suffers from wasting disease secondary to AIDS, and the step of administering treats wasting disease secondary to AIDS.

In further embodiments, the subject suffers from a malabsorption syndrome, and the step of administering treats the malabsorption syndrome. In yet further embodiments, the malabsorption syndrome is functional short bowel disease.

In still further embodiments, the step of administering reduces the incidence of a skin cancer (e.g., basal cell carcinoma, squamous cell carcinoma, or melanoma).

In other embodiments, the subject suffers from a sun burn, and the step of administering treats at least one symptom of the sun burn.

In yet other embodiments, the subject suffers from a hangover, and the step of administering treats the hangover.

In still other embodiments, the step of administering improves an energy state of the subject.

In certain embodiments, the step of administering improves alertness and cognition of the subject.

In particular embodiments, the step of administering slows aging of the subject.

In some embodiments, the step of administering increases rate of ATP production in the subject.

In further embodiments, the step of administering increases hydration of the subject.

In yet further embodiments, the step of administering enhances cellular repair mechanisms after exposure to UV radiation or sunlight.

In still embodiments, the step of administering enhances the benefits of physical therapy.

In other embodiments, the step of administering enhances the release of neurotransmitters releasable by a process requiring energy in the form of ATP.

In yet other embodiments, the step of administering enhances the delivery of pharmaceutical drugs and natural products to the subject.

In still other embodiments, the step of administering reduces the body mass index of the subject.

In some embodiments, the comestible composition is administered daily for at least 7 days. In particular embodiments, the comestible composition is administered daily for at least 15 days. In certain embodiments, the comestible composition is administered daily for 15 days or fewer.

In further embodiments, the comestible composition is administered once daily, twice daily, or three times daily.

In yet further embodiments, one serving of the comestible composition is administered per administration event.

### Definitions

The term “alertness,” as used herein, refers to the sensory awareness of a subject to the environment. Alertness may be measured in a subject through the subject's self-assessment, where the subject is asked to rate the subject's alertness on a scale of 1 to 10 with or without (e.g., before or after) the treatment with a composition. On this scale, 1 indicates low alertness and 10 indicates high alertness of the subject. In some embodiments, administration of a composition of the invention provides an increase in the subject's alertness rating of at least 2 on the alertness scale.

The term “athletic performance enhancement,” as used herein, refers to the subject's ability to perform certain athletic tasks. A non-limiting example of athletic performance enhancement includes the subject's ability to run faster and/or run longer following a period of treatment with a composition relative to that without a composition of the invention.

The term “cancer,” as used herein, refers to a class of malignant proliferative diseases. Non-limiting examples of cancers include pancreatic cancer and skin cancer (e.g., basal cell carcinoma or squamous cell carcinoma).

The term “cognition,” as used herein, refers to a subject's ability to perform certain mental tasks, as measured through the subject's self-assessment, where the subject is asked to rate the subject's cognition on a scale of 1 to 10 with or without (e.g., before or after) the treatment with a composition. On this scale, 1 indicates low cognition and 10 indicates high cognition capabilities of the subject. In some embodiments, administration of a composition of the invention provides an increase in the subject's cognition rating of at least 2 on the cognition scale.

The term “comestible,” as used herein, refers to compositions acceptable for ingestion by a human, as may be determined by applicable authorities (e.g., U.S. Food and Drug Administration). A composition of the invention may be, e.g., a nutraceutical composition or dietary supplement.

The term “diabetes,” as used herein, refers to both type I diabetes (juvenile-onset or diabetes mellitus) and type II diabetes (adult-onset diabetes). The term diabetes is also meant to include those individuals designated as being “pre-diabetic” or indicated as having a propensity to develop type II diabetes based on the presentation of one or more (e.g., one, two, three, or four) of the following symptoms: increased weight (obesity), decreased insulin absorption or sensitivity (e.g., decrease by at least 5%, 10%, 15%, 20%, 25%, 30%, 35%, 40%, 45%, 50%, 55%, or 60%), elevated blood glucose levels (e.g., one or more blood glucose readings greater than 104 mg/dL), and increased glycated hemoglobin levels (e.g., HbA_{1C} greater than 7.0%). Treatment of diabetes may result in an increase (e.g., by at least 5%, 10%, 15%, 20%, 25%, 30%, 35%, 40%, 45%, 50%, 55%, 60%, 70%, 80%, 90%, or 95%) in insulin absorption or sensitivity, a decrease (e.g., by at least 5%, 10%, 15%, 20%, 25%, 30%, 35%, 40%, 45%, 50%, 55%, or 60%) in blood glucose levels in a subject or patient population having elevated blood glucose (e.g., one or more blood glucose readings greater than 104 mg/dL), and a decrease (e.g., by at least 5%, 10%, 15%, 20%, 25%, or 30%) in glycated hemoglobin levels in a subject or patient population having increased glycated hemoglobin levels (e.g., HbA_{1C} greater than 7.0%).

The term “energy state,” as used herein, refers to the capability of a subject to engage in physically straining activities, as measured through the subject's self-assessment, where the subject is asked to rate the subject's energy state on a scale of 1 to 10 with or without (e.g., before or after) the treatment with a composition. On this scale, 1 indicates low energy state and 10 indicates high energy state of the subject. In some embodiments, administration of a composition of the invention provides an increase in the subject's energy state rating of at least 2 on the cognition scale. A subject having a higher energy state can perform an athletic task with superior endurance and/or superior intensity relative to a subject having a lower energy state.

The term “enhance the delivery,” as used herein, refers to an observable increase in the oral bioavailability of a molecule co-administered with a composition of the invention relative to the bioavailability of the same molecule administered without a composition of the invention. The delivery enhancement may be measured as an increase (e.g., at least 5%, 10%, 20%, 30%, 40%, 50%, 60%, 70%, 80%, 90%, or 100% increase) in the oral bioavailability of the molecule. Oral bioavailability of a molecule may be measured using methods known in the art, e.g., by measuring the AUC_{inf} of the molecule in a subject.

The term “enhancement of muscle protein synthesis,” as used herein, refers to the increase in the rate of protein production in muscles of a subject following administration of a composition of the invention relative to the rate of protein production in muscles of a subject without a composition of the invention (e.g., prior to administration of the composition of the invention). Muscle protein synthesis rate may be measured as the rate of radioactive amino acid incorporation. Typically, ¹³C L-leucine is used for this purpose.

The term “fatty acid transport facilitator,” as used herein, refers to a compound facilitating the transport of long-chain fatty acids across the inner mitochondrial membrane for subsequent β-oxidation. Non-limiting examples of fatty acid transport facilitators include carnitine and its O-esters (e.g., acetyl-L-carnitine, isovaleryl-L-carnitine, and propionyl-L-carnitine).

The term “increase rate of ATP production,” as used herein, refers to an increase in at least one observable state directly correlated with the ATP production or to a decrease in at least one observable state inversely correlated with the ATP product. The states directly correlated with the ATP production include alertness and cognition. The states inversely correlated with the ATP production include an ischemic condition (e.g., ischemic heart disease and congestive heart failure), a lung disease (e.g., asthma and chronic pulmonary disease), peripheral artery disease, irritable bowel disease, DNA damage condition, neurodegenerative diseases (e.g., Parkinson's disease, Alzheimer's disease, and vascular dementia), and generalized fatigue. A non-limiting example of a DNA damage condition is a sun burn.

The term “inorganic selenium,” as used herein, refers to a selenium-containing inorganic salt. Inorganic selenium used in compositions of the invention are those selenium-containing inorganic salts deemed acceptable for ingestion by applicable authorities (e.g., U.S. Food and Drug Administration). Preferably, inorganic selenium is a selenium-containing inorganic sodium salt. Non-limiting examples of inorganic selenium include sodium selenite and sodium selenate.

The term “intense period of exercise,” as used herein, refers to physical exercises to exhaustion. Non-limiting examples of the intense period of exercise include long distance running, push-ups, pull-ups, leg raises, box jumps, swimming, running a marathon, running an ultramarathon, weight lifting, participation in a game of soccer, participation in a game of rugby, participation in a game of football, and triathlons.

The term “long hike,” as used herein, refers to a hike across natural terrain for a period of at least 6 hours or for a distance of 5 miles/day to 50 miles/day.

The term “long period in the field,” as used herein, refers to a period of a subject's physical exertion without rest and exceeding the subject's routine physical exertion time periods. Non-limiting examples of the long period in the field include a military operation and a long hike.

The term “military operation,” as used herein, refers to a military action requiring physical exertion to exhaustion or beyond and/or maintenance of alertness and/or cognition levels substantially at a level of having rested. Non-limiting examples of military operations include combat, patrols, and operation of military equipment (e.g., an aircraft).

The terms “mitochondrial anti-oxidant” and “electron flux facilitator,” as used interchangeably herein, refer to redox-active compounds capable of reducing the production of mitochondrial oxygen reactive species. Non-limiting examples of mitochondrial anti-oxidants/electron flux facilitators include coenzyme Q10 and ubiquinol 10.

The term “polyphenol,” as used herein, refers to phenolic acids, stilbenes, and flavonoids. Phenolic acids are known in the art. Non-limiting examples of phenolic acids include hydroxybenzoic acids and hydroxycinnamic acids. Stilbenes are known in the art. A non-limiting example of stilbenes is resveratrol. Flavonoids are known in the art. Non-limiting examples of flavonoids include an anthocyanidin, hesperetin, quercetin, and luteolin.

The term “potassium salt,” as used herein, is a salt including the potassium cation (K⁺). Non-limiting examples of potassium salts include potassium chloride, potassium bicarbonate, and potassium citrate.

The term “providing a health benefit,” as used herein, refers to the beneficial effect on the health of a subject observable in or by a subject following the consumption of a composition of the invention. In some instances, a health benefit may be observed in or by a subject following the consumption of a single serving of a composition of the invention. In other instances, a health benefit may become observable in or by a subject after daily consumption of the composition of the invention for at least 3 days (e.g., at least 15 days). A health benefit may be the treatment of a subject in need thereof (e.g., a subject suffering from a disease, disorder, or condition). Alternatively, a health benefit may be a beneficial effect observed in or by a healthy subject following the consumption of a composition of the invention. Non-limiting examples of the health benefits include, for example, an athletic performance enhancement; reduction in the recovery times following an intense period of exercise (e.g., the intense period of exercise is running a marathon, weight lifting, participation in a game of soccer, participation in a game of rugby, or participation in a game of football); an energy state improvement or maintenance during a long period in the field (e.g., the long period in the field is a military operation or a long hike); improvement of alertness in the subject; enhancement of muscle protein synthesis in the subject during a period of immobility; improvement of an energy state of the subject; improvement of alertness and cognition of the subject; slowing aging of the subject; increasing rate of ATP production in the subject; increasing hydration of the subject; enhancement of cellular repair mechanisms after exposure to UV radiation or sunlight; enhancement of the benefits of physical therapy; enhancement of the release of neurotransmitters releasable by a process requiring energy in the form of ATP; and/or enhancement of the delivery of pharmaceutical drugs and natural products to the subject.

The term “reduces the body mass index,” as used herein, refers to the reduction of the body mass index in a subject, e.g., by at least 1% (e.g., by at least 5%, 10%, 15%, 20%, 25%, 30%, 35%, 40%, 45%, 50%, 55%, or 60%). The subject may have a body mass index of at least 25 (e.g., 25 to 50) prior to the administration of the composition of the invention. The subject may suffer from obesity prior to the administration of the composition of the invention.

The term “reduction in the recovery times following an intense period of exercise,” as used herein, refers to the reduction in the time period from the intense period of exercise to a normal state in a subject administered a composition of the invention relative to that without a composition of the invention, as assessed by the subject. For example, muscle soreness, energy state, and/or strength in a subject may recover faster following an intense period of exercise, when a composition of the invention is coupled.

The term “serving,” as used herein, refers to the amount of a composition to be consumed by a subject in a single administration event. Servings of the compositions of the invention are typically compliant with the regulations of the applicable authorities (e.g., the U.S. Food and Drug Administration). In the United States of America, the applicable regulations governing “serving” sizes and labeling may be found in, e.g., 21 C.F.R. §§ 101.9 and 101.12. A typical serving size for the compositions of the invention in the powder form may be, e.g., 2.5 to 24 g (e.g., 8 or 16 g). Typically, 8 to 24 g (e.g., 8 to 16 g) of the composition of the invention are administered daily in doses divided across one, two, or three administration events. A typical serving size for the composition of the invention in the beverage form may be, e.g., 12 US fl oz.

The term “sodium salt,” as used herein, is a salt including the sodium cation (Na⁺). Non-limiting examples of sodium salts include sodium chloride and sodium bicarbonate.

The term “subject,” as used herein, represents a human. The subject may be one suffering from a disease, disorder, or condition as determined by a qualified professional (e.g., a doctor or a nurse practitioner) with or without known in the art laboratory test(s) of sample(s) from the patient. The subject may be of any age, e.g., an adolescent or an adult.

The term “substrate for ATP synthesis,” as used herein, refers to an ATP biosynthesis precursor. A non-limiting example of the substrate for ATP synthesis is D-ribose.

“Treatment” and “treating,” as used herein, refer to the medical management of a subject with the intent to improve, ameliorate, or stabilize a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); treatment designed to reduce the incidence of the disease, disorder, or condition; and supportive treatment (treatment employed to supplement another therapy).

Other features and advantages of the invention will be apparent from the following description of the preferred embodiments thereof, and from the claims.

### BRIEF DESCRIPTION OF THE DRAWINGS

FIG. 1 is a graph showing the responsiveness of three groups of individuals after 15 days of administration with composition 1. Group 1 received the composition once a day, Group 2 received the composition twice a day, and Group 3 received the composition three times a day.

FIG. 2 is a graph showing the responsiveness of three groups of individuals after 30 days of administration with composition 1. Group 1 received the composition once a day, Group 2 received the composition twice a day, and Group 3 received the composition three times a day.

FIG. 3 is a graph showing the overall responsiveness to composition 1 based on gender.

FIG. 4 is a graph showing the overall responsiveness to composition 1 based on age.

FIG. 5 is a graph showing the overall responsiveness to composition 1, evaluated by combined energy and alertness, after 15 and 30 days.

### DETAILED DESCRIPTION OF THE INVENTION

In general, the invention provides comestible compositions including a mitochondrial anti-oxidant/electron flux facilitator (e.g., coenzyme Q10 or ubiquinol 10), a fatty acid transport facilitator (e.g., acetyl-L-carnitine, propionyl-L-carnitine, or isovaleryl-L-carnitine), and a substrate for ATP synthesis (e.g., D-ribose). The invention also provides kits including the comestible compositions of the invention and methods of using the comestible compositions of the invention.

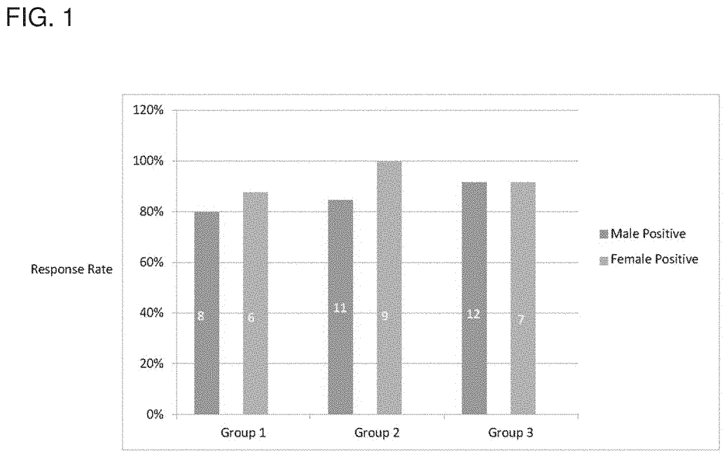

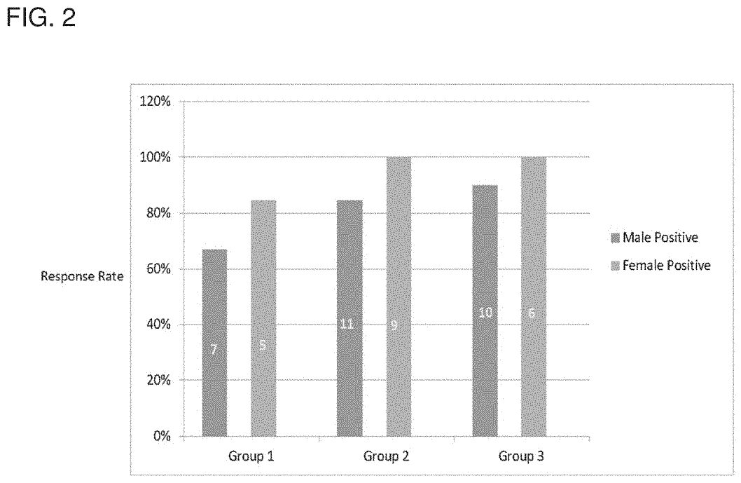

Advantageously, the combination of a mitochondrial anti-oxidant/electron flux facilitator (e.g., coenzyme Q10 or ubiquinol 10), a fatty acid transport facilitator (e.g., acetyl-L-carnitine, propionyl-L-carnitine, or isovaleryl-L-carnitine), and a substrate for ATP synthesis (e.g., D-ribose) may enhance ATP synthesis in a subject. For example, as described herein, subjects receiving an exemplary comestible composition of the invention responded to the comestible composition in a substantially dose-dependent manner (FIGS. 1 and 2). The exemplary composition containing D-Ribose, coenzyme Q10, and carnitine in the form of acetyl-L-carnitine was shown in a study to increase energy and alertness in normal subjects from all age groups independently of gender (Example 1). Biochemically it is difficult to quantify an increase in the absolute concentration of ATP in a cell resulting from a drug or a dietary supplement. A healthy cell utilizes ATP constantly to optimize the cells function. ATP is used as it is synthesized; it is not stored for later use. Thus the turnover and utilization of ATP or the rate of ATP synthesis and its use are physiologically relevant. This is the biochemical rationale for not measuring cellular ATP and, instead, assessing the consequence of enhanced energy production by the subject self-assessment for energy and alertness. By measuring a subject's energy at rest, during exercise, and as a consequence of a disease, and by measuring a subject's general alertness, the benefit of mitochondrial activation can be assessed.

Advantageously, the compositions and methods of the invention may enhance alertness and cognition and/or reduce fatigue in a subject without causing side effects associated with psychomotor stimulants (e.g., caffeine-based products), such as increasing heart rate or affecting sleep pattern. Because the compositions of the invention are psychomotor stimulant-free (e.g., caffeine-free), consumption of the compositions of the invention may result in the reduction or even cessation of the consumption of psychomotor stimulants (e.g., caffeine-based products) by the subject.

In the compositions, kits, and methods described herein, coenzyme Q10 may be replaced with a mitochondrial anti-oxidant/electron flux facilitator (e.g., ubiquinol 10). In the compositions, kits, and methods described herein, acetyl-L-carnitine may be replaced with a fatty acid transport facilitator (e.g., propionyl-L-carnitine or isovaleryl-L-carnitine). In the compositions, kits, and methods described herein, D-ribose may be replaced with another substrate for ATP synthesis.

Comestible Compositions

Comestible compositions of the invention include a mitochondrial anti-oxidant/electron flux facilitator (e.g., coenzyme Q10 or ubiquinol 10), a fatty acid transport facilitator (e.g., acetyl-L-carnitine, propionyl-L-carnitine, or isovaleryl-L-carnitine), and a substrate for ATP synthesis (e.g., D-ribose).

L-Carnitine (3-hydroxy-4-N-trimethylaminobuytrate) is a transporter of fatty acids into mitochondria for oxidation and ATP synthesis. Without adequate carnitine, ATP cannot be generated from fatty acids. Carnitine deficiency has been associated with insulin resistance, coronary artery disease, congestive heart failure, and aging (muscle weakness, mental health, reduced mobility, and/or diminished endurance).

Coenzyme Q10 (a.k.a. ubiquinone) is an anti-oxidant and a key component of the mitochondrial electron transport chain. Coenzyme Q10 help accelerate the synthesis of ATP and has been shown to improve physical exercise, treat heart failure and delay brain atrophy associated with Alzheimer's disease and Parkinson's disease.

D-Ribose, is a 5 carbon sugar with a negative glycemic index. Ribose is part of the ATP molecule and can be rate limiting in the synthesis of ATP. While ribose can be made endogenously, its rate of synthesis is often slower than needed for optimal ATP synthesis. Thus supplementation with ribose can accelerate the synthesis of ATP, particularly, under certain conditions, such as heart failure or as a result of intense exercise periods.

Preferably, the comestible composition of the invention includes the components at a pre-determined ratio. For example, the weight ratio of the substrate for ATP synthesis to the mitochondrial anti-oxidant (e.g., D-ribose to coenzyme Q10) may be, e.g., 25:1 to 100:1 (e.g., 50:1). The weight ratio of the fatty acid transport facilitator to the mitochondrial anti-oxidant (e.g., acetyl-L-carnitine to coenzyme Q10) may be, e.g., 5:2 to 10:1. The weight ratio of the fatty acid transport facilitator to the mitochondrial anti-oxidant (e.g., acetyl-L-carnitine to coenzyme Q10) may be, e.g., 5:1. The weight ratio of the substrate for ATP synthesis to the fatty acid transport facilitator (e.g., D-ribose to acetyl-L-carnitine) may be, e.g., 5:2 to 20:1 (e.g., 10:1). The weight ratio of the substrate for ATP synthesis to the fatty acid transport facilitator to the mitochondrial anti-oxidant (e.g., D-ribose to acetyl-L-carnitine to coenzyme Q10) may be, e.g., 50:5:1.

The compositions of the invention may be formulated for daily administration. Typically, the compositions of the invention may be formulated for administration once daily, twice daily, or three times daily. The comestible compositions are formulated for oral administration.

The compositions of the invention may be formulated to include an effective amount of each component. For example, the composition may include, e.g., 5 g to 16.5 g of the substrate for ATP synthesis (e.g., D-ribose) per serving (e.g., 5 g to 5.5 g of the substrate for ATP synthesis (e.g., D-ribose) per serving, 7.5 g to 8.3 g of the substrate for ATP synthesis (e.g., D-ribose) per serving, or 15 g to 16.5 g of the substrate for ATP synthesis (e.g., D-ribose) per serving). The composition may include, e.g., 500 mg to 1.65 g of the fatty acid transport facilitator (e.g., acetyl-L-carnitine) per serving (e.g., 500 mg to 550 mg of the fatty acid transport facilitator (e.g., acetyl-L-carnitine) per serving, 750 mg to 825 mg of the fatty acid transport facilitator (e.g., acetyl-L-carnitine) per serving, or 1.5 g to 1.65 g of the fatty acid transport facilitator (e.g., acetyl-L-carnitine) per serving). The composition may include, e.g., 100 mg to 300 mg of the mitochondrial anti-oxidant (e.g., coenzyme Q10) per serving (e.g., 100 mg to 110 mg of the mitochondrial anti-oxidant (e.g., coenzyme Q10) per serving, 200 mg to 220 mg of the mitochondrial anti-oxidant (e.g., coenzyme Q10) per serving, or 300 mg to 330 mg of the mitochondrial anti-oxidant (e.g., coenzyme Q10) per serving).

The effective amount of a given component per serving may relate to, e.g., the number of daily administration of the composition of the invention. For example, the composition formulated for administration three times daily may contain, e.g., 5 g to 5.5 g of the substrate for ATP synthesis (e.g., D-ribose) per serving. The composition formulated for administration three times daily may include, e.g., 500 mg to 550 mg of the fatty acid transport facilitator (e.g., acetyl-L-carnitine) per serving. The composition formulated for administration three times daily may include, e.g., 100 mg to 110 mg of the mitochondrial anti-oxidant (e.g., coenzyme Q10) per serving. The composition formulated for administration twice daily may include, e.g., 7.5 g to 8.3 g of the substrate for ATP synthesis (e.g., D-ribose) per serving. The composition formulated for administration twice daily may include, e.g., 750 mg to 825 mg of the fatty acid transport facilitator (e.g., acetyl-L-carnitine) per serving. The composition formulated for administration twice daily may include, e.g., 200 mg to 220 mg of the mitochondrial anti-oxidant (e.g., coenzyme Q10) per serving. The composition of formulated for administration once daily may include, e.g., 15 g to 16.5 g of the substrate for ATP synthesis (e.g., D-ribose) per serving. The composition formulated for administration once daily may include, e.g., 1.5 g to 1.65 g of the fatty acid transport facilitator (e.g., acetyl-L-carnitine) per serving. The composition formulated for administration once daily may include, e.g., 300 mg to 330 mg of the mitochondrial anti-oxidant (e.g., coenzyme Q10) per serving.

The composition of the invention may include an antioxidant. An antioxidant may enhance the health benefits of the composition of the invention further. Without wishing to be bound by theory, an antioxidant may be used to decrease reactive oxygen species in mitochondria, thereby enhancing the viability of mitochondria. Antioxidants are known in the art. Non-limiting examples of an antioxidant include pyrroloquinoline quinone (PQQ), vitamin C, vitamin E (e.g., a tocopherol, such as α-tocopherol, β-tocopherol, or γ-tocopherol, or a mixture thereof), β-carotene, a polyphenol (e.g., a phenolic acid, a stilbene, or a flavonoid), or an inorganic selenium. The composition of the invention may be formulated to provide the antioxidant at a dose of 1 to 100 mg/day.

The composition of the invention may include one or more Vitamins B (e.g., Vitamin B3, Vitamin B6, or Vitamin B12). The composition of the invention may include the one or more Vitamins B in an amount corresponding to 100% to 1000% of the recommended dietary allowance (RDA). Recommended dietary allowances for Vitamins B are known in the art. The recommended dietary allowances may be found, for example, in “Dietary Reference Intakes for Thiamin, Riboflavin, Niacin, Vitamin B6, Folate, Vitamin B12, Pantothenic Acid, Biotin, and Choline,” National Academy Press, Washington, D.C. (1998), the disclosure of which is incorporated herein. For example, RDA for Vitamin B3 may be, e.g., 14-18 mg/day; RDA for Vitamin B6 may be, e.g., 1.2-2.0 mg/day; and RDA for Vitamin B12 may be, e.g., 2.4-2.8 mg/day. The composition of the invention may be formulated to provide, e.g., 14 to 180 mg/day of Vitamin B3. The composition of the invention may be formulated to provide, e.g., 1.2 to 20 mg/day of Vitamin B6. The composition of the invention may be formulated to provide, e.g., 2.4 to 28 mg/day of Vitamin B12. A Vitamin B may be included in the compositions of the invention to improve further energy state-augmenting, fatigue-reducing, alertness-enhancing, and cognition-enhancing properties of the compositions of the invention.

The composition of the invention may include an NAD or a precursor of NAD (e.g., nicotinamide riboside). When nicotinamide riboside is used, the composition of the invention may be formulated to provide 300-1000 mg/day (e.g., 500±100 mg/day) of nicotinamide riboside.

The composition of the invention may include a flavoring (e.g., a citrus punch flavoring or mojito flavoring). The flavoring may include, e.g., citric acid. The flavoring may be included in the compositions of the invention to enhance the palatability of the composition of the invention and/or to mask the taste of at least some of the components. The composition of the invention may include, e.g., 2 to 2.5 grams of flavoring per serving.

The composition of the invention may include a sodium salt and/or potassium salt. The sodium and/or potassium salts may be included in the compositions of the invention to enhance the hydration effect of the compositions of the invention. Non-limiting examples of sodium salts include sodium chloride and sodium bicarbonate. Non-limiting examples of potassium salts include potassium chloride, potassium bicarbonate, and potassium citrate. In particular, the composition of the invention including a sodium and/or potassium salts may be a hydration beverage or a powder, which, upon mixing with water, may produce a hydration beverage. The composition of the invention may include the sodium salt in an amount providing 200 mg to 800 mg of Na⁺ per serving (e.g., 200 mg to 240 mg of Na⁺ per serving, 300 mg to 400 mg of Na⁺ per serving, or 600 mg to 800 mg of Na⁺ per serving). Additionally or alternatively, the composition of the invention may include a potassium salt in an amount providing 150 mg to 500 mg of K⁺ per serving (e.g., 150 mg to 165 mg of K⁺ per serving, 225 mg to 250 mg of K⁺ per serving, or 450 mg to 500 mg of K⁺ per serving).

The amount of the sodium and/or potassium salts per serving may relate to, e.g., the number of daily administration of the composition of the invention. For example, the composition of the invention formulated for three times daily administration may include, e.g., the sodium salt in an amount providing 200 mg to 240 mg of Na⁺ per serving. The composition of the invention formulated for three times daily administration may include, e.g., the potassium salt in an amount providing 150 mg to 165 mg of K⁺ per serving. The composition of the invention formulated for twice daily administration may include, e.g., the sodium salt in an amount providing 300 mg to 400 mg of Na⁺ per serving. The composition of the invention formulated for twice daily administration may include, e.g., the potassium salt in an amount providing 225 mg to 250 mg of K⁺ per serving. The composition of the invention formulated for once daily administration may include, e.g., the sodium salt in an amount providing 600 mg to 800 mg of Na⁺ per serving. The composition of the invention formulated for once daily administration may include, e.g., the potassium salt in an amount providing 450 mg to 500 mg of K⁺ per serving.

The composition of the invention may include silicon dioxide (e.g., 1% to 2% (w/w) of silicon dioxide). The composition of the invention may include silicon dioxide as a drying agent to enhance storage stability of the powder compositions of the invention.

The composition of the invention may be formulated as a powder or a beverage (e.g., a hydration beverage). The beverage may contain, e.g., 12 US fl oz of water per serving.

Kits

The invention provides kits including a comestible composition of the invention (e.g., a powder) and instructions to mix the composition with water (e.g., with 12 US fl oz of water) to produce a beverage. The instructions may further include an instruction to consume the beverage within 1 hour of the mixing step. The instructions may further include an instruction to consume the beverage once daily, twice daily, or three times daily.

Methods of Preparing a Beverage

The invention provides a method for preparing a beverage of the invention. The method includes mixing a comestible composition of the invention with water (e.g., with 12 US fl oz of water) to produce a beverage.

Health Benefits

The invention also provides methods of providing a health benefit to a subject (e.g., a subject in need thereof). These methods of the invention typically include administering the comestible composition of the invention to the subject (e.g., a subject in need thereof). In some instances, the composition of the invention is administered to the subject for at least 3 days (e.g., at least 15 days). In further instances, the composition of the invention is administered to the subject for 15 days of fewer (e.g., 30 days or fewer), e.g., to provide the health benefit. The composition of the invention may be administered once daily, twice daily, or three times daily. The subject may consume, e.g., one serving of the comestible composition per administration event. The step of administering the composition of the invention to the subject may include the subject self-administering the composition of the invention. Alternatively, the composition of the invention may be administered to the subject by an attendant caretaker or nurse.

Without wishing to be bound by theory, the compositions of the invention may provide the health benefits described herein by increasing the rate of the ATP synthesis in a subject.

The health benefit may include an athletic performance enhancement; reduction in the recovery times following an intense period of exercise (e.g., the intense period of exercise is running a marathon, weight lifting, participation in a game of soccer, participation in a game of rugby, or participation in a game of football); an energy state improvement or maintenance during a long period in the field (e.g., the long period in the field is a military operation or a long hike); improvement of alertness in the subject; enhancement of muscle protein synthesis in the subject during a period of immobility; or reduction of the body mass index of a subject. Protein synthesis requires ATP, each amino acid added to a growing protein chain requires minimally four ATP molecules. Accordingly, without wishing to be bound by theory, the ATP production increase following administration of the composition of the invention may enhance muscle protein synthesis.

Alternatively, the health benefit may be the treatment of a subject in need thereof (e.g., the treatment of a disease, disorder, or condition in a subject). In some instances, the disease, disorder, or condition is associated with an inadequate production of ATP (e.g., the disease, disorder, or condition is associated with mitochondrial dysfunction). In further instances the disease, disorder, or condition is Alzheimer's Disease, Parkinson's Disease, Huntington's Disease, amyotrophic lateral sclerosis, cardiovascular disease including congestive heart failure and atherosclerosis, metabolic diseases such as diabetes, neuroendocrine disorders, multiple sclerosis, psychiatric diseases such as depression, autism, bipolar syndromes, schizophrenia, chronic fatigue syndromes, Gulf War illnesses, fibromyalgia, or chronic muscle atrophy. In particular instances, the disease, disorder, or condition is ischemic heart disease, congestive heart failure, chronic fatigue syndrome, or peripheral arterial disease. In further instances, the disease, disorder, or condition is amyotrophic lateral sclerosis, myasthenia, diabetes (e.g., type I diabetes or type II diabetes), Parkinson's disease, Huntington's disease, or Alzheimer's disease. In the subject suffering from Parkinson's disease, the step of administering the composition of the invention may slow the degeneration of dopaminergic neurons. In the subject suffering from Alzheimer's disease, the step of administering the composition of the invention may slow the degeneration of cholinergic neurons. In further instances, the disease, disorder, or condition is a hypothyroid condition associated with energy and metabolic deficiencies. In certain instances, the disease, disorder, or condition is wasting disease secondary to cancer (e.g., pancreatic cancer). In some instances, the disease, disorder, or condition is wasting disease secondary to AIDS. In further instances, the disease, disorder, or condition is a malabsorption syndrome (e.g., functional short bowel disease). In particular instances, the step of administering reduces the incidence of a skin cancer (e.g., basal cell carcinoma or squamous cell carcinoma). In certain instances, the disease, disorder, or condition is a sun burn. In yet further instances, the disease, disorder, or condition is a hangover.

Additionally or alternatively to the above, the methods described herein may provide further health benefits. For example, the step of administering may, e.g., improve an energy state of the subject, improve alertness and cognition of the subject, increase rate of ATP production in the subject.

The step of administering may slow aging of the subject. Cellular aging may be measured by a number of criteria, including enhances anabolic events such as increased muscle mass or reversing or stabilizing neuronal degradation or stabilizing or reversing any chronic disease state. Healthy cellular functions translate into health bodies affecting longevity.

The step of administering may increase hydration of the subject. A hydration state of the subject may be assessed through eye examination. Alternatively, hydration state may be assessed using urine flow and/or electrolyte composition and osmolarity of the urine.

The step of administering may enhance cellular repair mechanisms after exposure to UV radiation or sunlight. Without wishing to be bound by theory, the enhancement cellular repair mechanisms after exposure to UV radiation or sunlight may be achieved through stimulation of the ATP production in a subject using a composition of the invention. UV light is known to cause cellular damage and affect DNA, and cellular repair requires ATP. Examples of cellular repair mechanisms include repairing ion gradients to restore the cell membrane potential to a normal level (approximately −90 mv), preventing cellular apoptosis in damaged cells, and DNA repair.

The step of administering may enhance the benefits of physical therapy. Physical therapy is typically prescribed when subject's movement is impaired by a surgery, accident, or exercise. For example, when a leg or arm is fractured and a cast is applied, muscle synthesis is reduced and muscle catabolism is increased, resulting in muscle mass lost. Without wishing to be bound by theory, the enhancement of the physical therapy benefits may be achieved through stimulation of the ATP production in a subject using a composition of the invention. This is because ATP is required for muscle synthesis, and, therefore, increase in the ATP production may increase muscle synthesis or reduce muscle loss during periods of immobility. Thus, recovery times may be enhanced and muscle mass and strength increased.

The step of administering may enhance the release of neurotransmitters releasable by a process requiring energy in the form of ATP. Typically, neurotransmitters are released from nerve endings by exocytosis, which requires ATP. Without wishing to be bound by theory, the enhancement of the neurotransmitters release may be achieved through stimulation of ATP production using a composition of the invention. Neurotransmitter release may be measured by the presence of neurotransmitter metabolites in the blood or in the urine. In the case of catecholamines, products of monoamine oxidase may be measured. In the case of acetylcholine, products of cholineresterase may be measured.

The step of administering may enhance the delivery of pharmaceutical drugs and natural products to the subject. Upon administration to a subject, a composition of the invention may improve gastrointestinal absorption of molecules (e.g., pharmaceutical drugs or natural products) in the subject. Without wishing to be bound by theory, the improvement in the gastrointestinal absorption of molecules may result from the composition of the invention enhancing the activity of ATP-dependent ABC transporters in cells lining the GI tract of a subject. Typically, these ATP-dependent ABC transporters are responsible for the gastrointestinal absorption of L-amino acids (e.g., arginine, glutamine, and histidine).

### EXAMPLES

The following is an example of the methods and compositions of the invention. It is understood that various other embodiments may be practiced, given the description provided herein.

### Example 1. Effectiveness of Composition 1

The effect of administration of composition 1 (D-ribose, acetyl-L-carnitine, and coenzyme Q10 in a ratio of 50:5:1, respectively) was studied in healthy individuals between 22 and 84 years of age. No one component of this mixture may be as effective as the combination of all three, particularly, at the specific ratio and strength tested.

The serving size of composition 1 used in the tests described herein was about 8 g (5.5 g of D-ribose, 550 mg of acetyl-L-carnitine, 110 mg coenzyme Q10, and 2.5 g of a citrus punch or mojito flavoring agent). The test subjects were instructed to mix powdered composition 1 with 12 US fl oz of water and consume the resulting mixture within one hour of mixing.

Effectiveness of composition 1 in the improvement of energy and alertness was determined by functional assays rather than biochemical assays, as the absolute steady state concentrations of ATP in cells may or may not change, even if the rate of synthesis will change. This is because as ATP is made, it is utilized to improve cell function and cell repair.

Of the total individuals in the study, 60% were men and 40% were women. 50% were over 40 years of age and 50% were under 40 years of age. In total, 62 individuals completed a 30 day trial period with energy and alertness evaluations at 15 and 30 days after dosing. Energy and alertness evaluations were assessed using a scale of 1 to 10, where 1 indicated minimal energy and alertness and 10 indicated maximum energy and alertness. The response rate was measured as the percent of individuals experiencing a positive response after administration of the composition.

FIG. 1 shows the responsiveness of individuals after 15 days of administration with the composition. It was observed that male and female responses to the composition were similar. Group 1 was administered the composition once a day, Group 2 was administered the composition twice a day, and Group 3 was administered the composition three times a day. The composition was highly effective after daily dosing for 15 days. The intensity of the response increased with increases in daily doses, i.e., Group 3 response was greater than Group 2, which was greater than Group 1. In Group 1, 5 of 19 were non responders; in Group 2, 2 of 22 were non responders; and in Group 3, 2 of 21 were non responders. In total, 62 individuals completed the 15-day assessment.

Furthermore, as depicted in FIG. 2, the highly effective response documented at 15 days remained and increased slightly after 30 days of continuous daily dosing, indicating no tolerance to the positive effect of the combination. In total, 56 individuals completed the 30-day assessment.

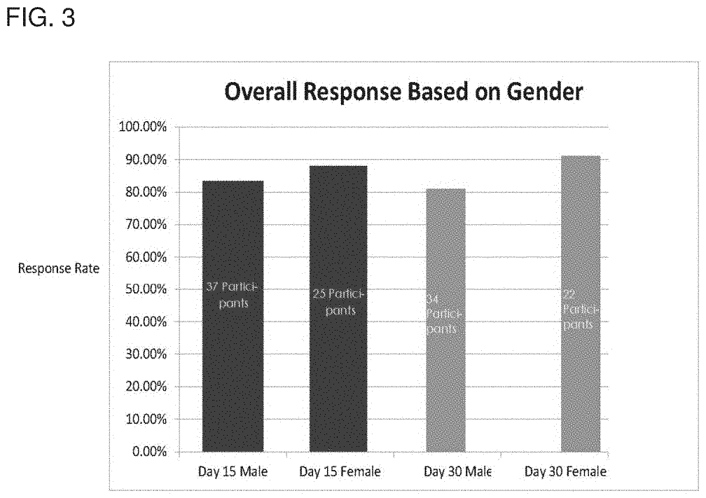

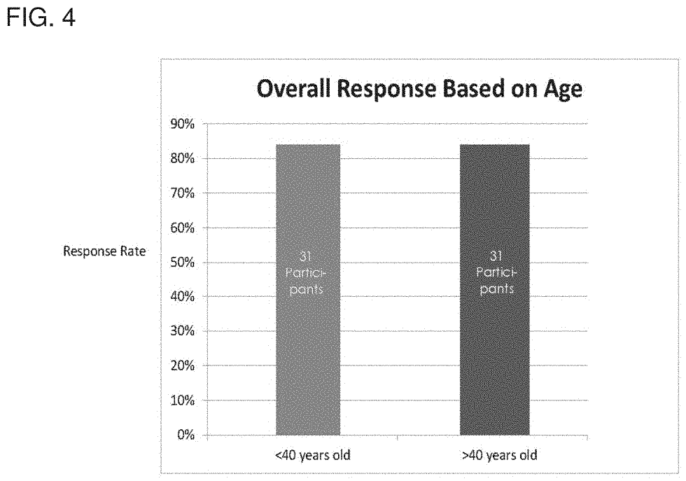

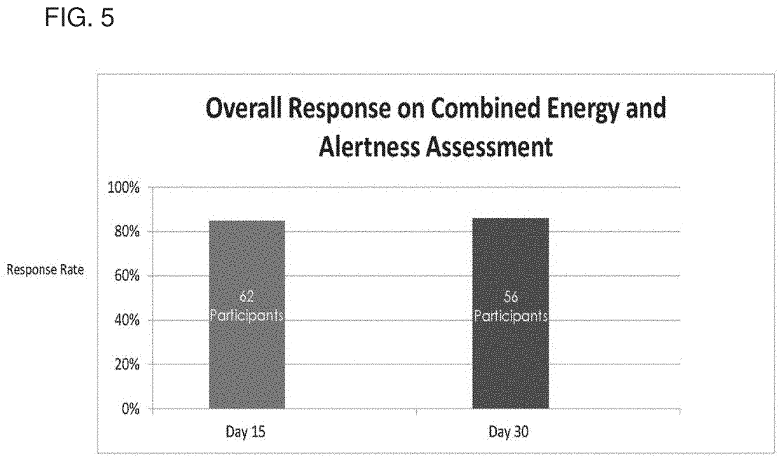

At both 15 days and 30 days, there was no evidence of a gender preference in test subjects, including post-menopausal women (FIG. 3). Moreover, the composition showed effectiveness for both individuals under 40 years of age and over 40 years of age (FIG. 4). Finally, it was shown that the composition was equally effective at enhancing alertness and cognition as enhancing energy (FIG. 5).

Several additional observations were documented during the study. It was observed that over 58% of individuals noted that their use of caffeine products diminished by about 50% after taking the composition. After the 30-day trial period and ceasing daily administration of the composition, there was a loss of energy and the onset of fatigue. Furthermore, it was observed that, unlike caffeine, the product neither affected sleep patterns nor raised heart rate.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A comestible composition comprising a substrate for ATP synthesis, a mitochondrial anti-oxidant, and a fatty acid transport facilitator.

2. The comestible composition of claim 1, wherein the weight ratio of the substrate for ATP synthesis to the mitochondrial anti-oxidant is 25:1 to 100:1.

3. The comestible composition of claim 2, wherein the weight ratio of the substrate for ATP synthesis to the mitochondrial anti-oxidant is 50:1.

4. The comestible composition of any one of claims 1 to 3, wherein the weight ratio of the fatty acid transport facilitator to the mitochondrial anti-oxidant is 5:2 to 10:1.

5. The comestible composition of claim 4, wherein the weight ratio of the fatty acid transport facilitator to the mitochondrial anti-oxidant is 5:1.

6. The comestible composition of any one of claims 1 to 5, wherein the weight ratio of the substrate for ATP synthesis to the fatty acid transport facilitator is 5:2 to 20:1.

7. The comestible composition of claim 6, wherein the weight ratio of the substrate for ATP synthesis to the fatty acid transport facilitator is 10:1.

8. The comestible composition of claim 1, wherein the weight ratio of the substrate for ATP synthesis to the fatty acid transport facilitator to the mitochondrial anti-oxidant is 50:5:1.

9. The comestible composition of any one of claims 1 to 8, wherein the composition is formulated for administration once daily, twice daily, or three times daily.

10. The comestible composition of any one of claims 1 to 9, wherein the composition comprises 5 g to 16.5 g of the substrate for ATP synthesis per serving.

11. The comestible composition of claim 10, wherein the composition is formulated for administration three times daily, and the composition comprises 5 g to 5.5 g of the substrate for ATP synthesis per serving.

12. The comestible composition of claim 10, wherein the composition is formulated for administration twice daily, and the composition comprises 7.5 g to 8.3 g of the substrate for ATP synthesis per serving.

13. The comestible composition of claim 10, wherein the composition is formulated for administration once daily, and the composition comprises 15 g to 16.5 g of the substrate for ATP synthesis per serving.

14. The comestible composition of any one of claims 1 to 9, wherein the composition comprises 500 mg to 1.65 g of acetyl-L-carnitine per serving.

15. The comestible composition of claim 14, wherein the composition is formulated for administration three times daily, and the composition comprises 500 mg to 550 mg of the fatty acid transport facilitator per serving.

16. The comestible composition of claim 14, wherein the composition is formulated for administration twice daily, and the composition comprises 750 mg to 825 mg of the fatty acid transport facilitator per serving.

17. The comestible composition of claim 14, wherein the composition is formulated for administration once daily, and the composition comprises 1.5 g to 1.65 g of the fatty acid transport facilitator per serving.

18. The comestible composition of any one of claims 1 to 9, wherein the composition comprises 100 mg to 300 mg of the mitochondrial anti-oxidant per serving.

19. The comestible composition of claim 18, wherein the composition is formulated for administration three times daily, and the composition comprises 100 mg to 110 mg of the mitochondrial anti-oxidant per serving.

20. The comestible composition of claim 18, wherein the composition is formulated for administration twice daily, and the composition comprises 200 mg to 220 mg of the mitochondrial anti-oxidant per serving.

21. The comestible composition of claim 18, wherein the composition is formulated for administration once daily, and the composition comprises 300 mg to 330 mg of the mitochondrial anti-oxidant per serving.

22. The comestible composition of any one of claims 1 to 21, further comprising an antioxidant.

23. The comestible composition of claim 22, wherein the antioxidant is pyrroloquinoline quinone (PQQ), vitamin C, vitamin E, β-carotene, a polyphenol, or an inorganic selenium.

24. The comestible composition of claim 23, wherein the composition is formulated to provide the antioxidant at a dose of 1 to 100 mg/day.

25. The comestible composition of any one of claims 1 to 24, further comprising one or more Vitamins B.

26. The comestible composition of claim 25, wherein the one or more Vitamins B are selected from the group consisting of Vitamin B3, Vitamin B6, and Vitamin B12.

27. The comestible composition of claim 25 or 26, wherein the one or more Vitamins B are present in an amount corresponding to 100% to 1000% of the recommended dietary allowance.

28. The comestible composition of any one of claims 1 to 27, further comprising NAD or an NAD precursor.

29. The comestible composition of claim 28, wherein the NAD precursor is nicotinamide riboside.

30. The comestible composition of any one of claims 1 to 29, further comprising a flavoring.

31. The comestible composition of claim 30, wherein the composition comprises 2 to 2.5 grams of flavoring per serving.

32. The comestible composition of claim 31, wherein the flavoring is a citrus punch flavoring or mojito flavoring.

33. The comestible composition of any one of claims 30 to 32, wherein the flavoring comprises citric acid.

34. The comestible composition of any one of claims 1 to 33, further comprising a sodium salt.

35. The comestible composition of claim 34, wherein the composition comprises the sodium salt in an amount providing 200 mg to 800 mg of Na⁺ per serving.

36. The comestible composition of claim 35, wherein the composition comprises the sodium salt in an amount providing 200 mg to 240 mg of Na⁺ per serving, and the composition is formulated for three times daily administration.

37. The comestible composition of claim 35, wherein the composition comprises the sodium salt in an amount providing 300 mg to 400 mg of Na⁺ per serving, and the composition is formulated for twice daily administration.

38. The comestible composition of claim 35, wherein the composition comprises the sodium salt in an amount providing 600 mg to 800 mg of Na⁺ per serving, and the composition is formulated for once daily administration.

39. The comestible composition of any one of claims 1 to 38, further comprising a potassium salt.

40. The comestible composition of claim 39, wherein the composition comprises the potassium salt in an amount providing 150 mg to 500 mg of K⁺ per serving.

41. The comestible composition of claim 39, wherein the composition comprises the potassium salt in an amount providing 150 mg to 165 mg of K⁺ per serving, and the composition is formulated for three times daily administration.

42. The comestible composition of claim 39, wherein the composition comprises the potassium salt in an amount providing 225 mg to 250 mg of K⁺ per serving, and the composition is formulated for twice daily administration.

43. The comestible composition of claim 39, wherein the composition comprises the potassium salt in an amount providing 450 mg to 500 mg of K⁺ per serving, and the composition is formulated for once daily administration.

44. The comestible composition of any one of claims 1 to 43, further comprising silicon dioxide.

45. The comestible composition of claim 44, wherein the composition comprises 1% to 2% (w/w) of silicon dioxide.

46. The comestible composition of any one of claims 1 to 45, wherein the mitochondrial anti-oxidant is coenzyme Q10.

47. The comestible composition of any one of claims 1 to 45, wherein the mitochondrial anti-oxidant is ubiquinol 10.

48. The comestible composition of any one of claims 1 to 47, wherein the fatty acid transport facilitator is acetyl-L-carnitine.

49. The comestible composition of any one of claims 1 to 47, wherein the fatty acid transport facilitator is propionyl-L-carnitine.

50. The comestible composition of any one of claims 1 to 47, wherein the fatty acid transport facilitator is isovaleryl-L-carnitine.

51. The comestible composition of any one of claims 1 to 50, wherein the substrate for ATP synthesis facilitator is D-ribose.

52. The comestible composition of any one of claims 1 to 51, wherein the composition is a powder.

53. The comestible composition of any one of claims 1 to 51, wherein the composition is a beverage.

54. The comestible composition of claim 53, wherein the beverage is a hydration beverage.

55. A kit comprising the comestible composition of claim 52 and instructions to mix one serving of the composition with water to produce a beverage.

56. The kit of claim 55, wherein instructions comprise an instruction to mix one serving of the composition with 12 US fl oz of water.

57. The kit of claim 55 or 56, wherein the instructions further comprise instructions to consume the beverage within 1 hour of the mixing step.

58. The kit of any one of claims 55 to 57, wherein the instructions further comprise instructions to consume the beverage once daily, twice daily, or three times daily.

59. A method of preparing the composition of claim 53 or 54 comprising mixing the comestible composition of claim 52 with water.

60. The method of claim 59, wherein the comestible composition of claim 52 is mixed with about 12 US fl oz of water.

61. A method of providing a health benefit to a subject, the method comprising administering the comestible composition of any one of claims 1 to 54 to the subject.

62. The method of claim 61, wherein the subject is in need of the health benefit.

63. The method of claim 61 or 62, wherein the health benefit comprises an athletic performance enhancement.

64. The method of any one of claims 61 to 63, wherein the health benefit comprises reduction in the recovery times following an intense period of exercise.

65. The method of claim 61, wherein the intense period of exercise is running a marathon, weight lifting, participation in a game of soccer, participation in a game of rugby, or participation in a game of football.

66. The method of any one of claims 61 to 65, wherein the health benefit comprises an energy state improvement or maintenance during a long period in the field.

67. The method of claim 66, wherein the long period in the field is a military operation or a long hike.

68. The method of claim 61, wherein the health benefit comprises improvement of alertness in the subject.

69. The method of claim 61, wherein the health benefit comprises enhancement of muscle protein synthesis in the subject during a period of immobility.

70. A method of treating a subject in need thereof, the method comprising administering the comestible composition of any one of claims 1 to 53 to the subject.

71. The method of claim 70, wherein the subject suffers from a disease, disorder, or condition associated with mitochondrial dysfunction, and the step of administering treats the disease, disorder, or condition.

72. The method of claim 70, wherein the subject suffers from a disease, disorder, or condition associated with an inadequate production of ATP, and the step of administering treats the disease, disorder, or condition.

73. The method of claim 70, wherein the subject suffers from a disease, disorder, or condition selected from the group consisting of ischemic heart disease, congestive heart failure, chronic fatigue syndrome, and peripheral arterial disease, and the step of administering treats the disease, disorder, or condition.

74. The method of claim 70, wherein the subject suffers from a disease, disorder, or condition selected from the group consisting of amyotrophic lateral sclerosis, myasthenia, diabetes, Parkinson's disease, Huntington's disease, and Alzheimer's disease, and the step of administering treats the disease, disorder, or condition.

75. The method of claim 74, wherein the disease, disorder, or condition is Parkinson's disease.

76. The method of claim 74 or 75, wherein the step of administering slows the degeneration of dopaminergic neurons in the subject.

77. The method of claim 74, wherein the disease, disorder, or condition is Alzheimer's disease.

78. The method of claim 74 or 77, wherein the step of administering slows the degeneration of cholinergic neurons in the subject.

79. The method of claim 70, wherein the subject suffers from hypothyroidism associated with energy and metabolic deficiencies, and the step of administering treats hypothyroidism.

80. The method of claim 70, wherein the subject suffers from wasting disease secondary to cancer, and the step of administering treats wasting disease secondary to cancer.

81. The method of claim 80, wherein the wasting disease is secondary to pancreatic cancer.

82. The method of claim 70, wherein the subject suffers from wasting disease secondary to AIDS, and the step of administering treats wasting disease secondary to AIDS.

83. The method of claim 70, wherein the subject suffers from a malabsorption syndrome, and the step of administering treats the malabsorption syndrome.

84. The method of claim 70, wherein the malabsorption syndrome is functional short bowel disease.

85. The method of claim 70, wherein the step of administering reduces the incidence of a skin cancer.

86. The method of claim 85, wherein the skin cancer is basal cell carcinoma, squamous cell carcinoma, or melanoma.

87. The method of claim 70, wherein the subject suffers from a sun burn, and the step of administering treats at least one symptom of the sun burn.

88. The method of claim 70, wherein the subject suffers from a hangover, and the step of administering treats the hangover.

89. The method of any one of claims 61 to 88, wherein the step of administering improves an energy state of the subject.

90. The method of any one of claims 61 to 89, wherein the step of administering improves alertness and cognition of the subject.

91. The method of any one of claims 61 to 90, wherein the step of administering slows aging of the subject.

92. The method of any one of claims 61 to 91, wherein the step of administering increases rate of ATP production in the subject.

93. The method of any one of claims 61 to 92, wherein the step of administering increases hydration of the subject.

94. The method of any one of claims 61 to 93, wherein the step of administering enhances cellular repair mechanisms after exposure to UV radiation or sunlight.

95. The method of any one of claims 61 to 94, wherein the step of administering enhances the benefits of physical therapy.

96. The method of any one of claims 61 to 95, wherein the step of administering enhances the release of neurotransmitters releasable by a process requiring energy in the form of ATP.

97. The method of any one of claims 61 to 96, wherein the step of administering enhances the delivery of pharmaceutical drugs and natural products to the subject.

98. The method of any one of claims 61 to 97, wherein the step of administering reduces the body mass index of the subject.

99. The method of any one of claims 61 to 98, wherein the comestible composition is administered daily for at least 7 days.

100. The method of any one of claims 61 to 99, wherein the comestible composition is administered daily for at least 15 days.

101. The method of any one of claims 61 to 100, wherein the comestible composition is administered daily for 15 days or fewer.

102. The method of any one of claims 61 to 101, wherein the comestible composition is administered once daily, twice daily, or three times daily.

103. The method of claim 102, wherein one serving of the comestible composition is administered per administration event.
